(19) United States
(12) Patent Application Publication
(10) Pub. No.: US 2019/0287676 A1
(43) Pub. Date: Sep. 19, 2019

(54) TRACKING, COMPARISON, AND ANALYTICS OF ACTIVITIES, FUNCTIONS, AND OUTCOMES IN PROVISION OF HEALTHCARE SERVICES

(71) Applicant: SirenMD, Miami, FL (US)

(72) Inventors: Lee D. Kaplan, Miami, FL (US); Andrew Willert, Parkland, FL (US)

(73) Assignee: SirenMD, Miami, FL (US)

(21) Appl. No.: 16/353,259

(22) Filed: Mar. 14, 2019

Related U.S. Application Data
(60) Provisional application No. 62/643,572, filed on Mar. 15, 2018.

(51) Int. Cl. G16H 40/20; G06Q 10/06; G06F 3/0482

(52) U.S. Cl. CPC G16H 40/20; G06F 3/0482; G06Q 10/063114

(57) ABSTRACT

Methods and systems for tracking and analyzing activities, functions, and outcomes in the provision of healthcare services are disclosed. Various types of data from multiple sensors and devices can be captured and analyzed. Metrics have more value because they are not considered in a vacuum, but rather compared with those of other providers. Caregivers are provided user interfaces with time spent engaging in particular activities and how that compares with other healthcare professionals.

### CROSS-REFERENCE TO RELATED APPLICATION

This application claims priority to U.S. Provisional Patent Application No. 62/643,572, entitled “TRACKING, COMPARISON, AND ANALYTICS OF ACTIVITIES, FUNCTIONS, AND OUTCOMES IN PROVISION OF HEALTHCARE SERVICES,” filed Mar. 15, 2018, the entirety of which is incorporated herein by reference.

### BACKGROUND

Healthcare providers, in caring for patients, perform a great variety of functions and activities, and only certain activities are commonly tracked. For example, a healthcare provider may perform a surgical procedure and/or administer a treatment on a certain day for a given patient, and the procedure/treatment is recorded in the patient's medical record. The services provided can be assigned a metric in terms of predetermined value (based on, e.g., relative value units, or RVUs, used by the U.S. Department of Health and Human Services). But this does not fully consider the effort expended by the provider. Activities of healthcare providers, other than performing procedures and administering treatments, tend to be poorly tracked and accounted for in delivery of care.

For example, the time spent by a provider in such activities as reviewing case files and medical literature, scheduling meetings and interventions, speaking on the telephone, messaging via electronic mail (e-mail) or text (such as SMS or via other messaging applications), preparing for and performing services, and/or consulting with colleagues, patients, and the family of patients, although an essential part of the delivery of healthcare services is not traditionally tracked, assigned a separate value, or otherwise effectively captured in medical records. Moreover, such metrics, even if systematically tracked, are not compared with those of other healthcare providers and organizations.

What are needed are systems and methods that address one or more of the above, as well as other, shortcomings of conventional approaches.

### SUMMARY

In accordance with at least some aspects of the present disclosure, methods and systems for tracking and analyzing activities, functions, and outcomes in the provision of healthcare services are disclosed. Metrics have more value because they are not considered in a vacuum, but rather compared with those of other providers. For example, if a provider is aware of the time spent engaging in particular activities and how that compares with other healthcare professionals, the provider can, for example, better evaluate his or her performance, accumulate evidence for value added to a practice (which might be useful in negotiations with practices groups and organizations), and/or determine whether he or she could benefit from additional tools (such as new technologies that could help the provider enhance efficiency, outcomes, and/or revenue). Further, knowledge of how the providers in a healthcare practice (e.g., a clinic, group, hospital, etc.) fare relative to providers in other organizations would allow the organization to determine whether and how it could enhance performance by targeting certain identified metrics. Furthermore, comparison data will allow caregivers to compete with each other to enhance their overall statistics or their statistics in a future time period.

One or more embodiments of the disclosure relate to a system. The system may be configured to acquire data on one or more activities of one or more caregivers in a healthcare organization. The data may be acquired by a data acquisition engine of the system. The system may also be configured to generate comparison data on the caregivers in the healthcare organization. The comparison data may be generated by an analytics engine of the system. The system may moreover be configured to generate one or more user interfaces. The user interfaces may include data on activities of the provider. The user interfaces may alternatively or additionally include comparison data comparing one or more metrics of multiple providers. The user interfaces may be generated by a data presentation engine of the system.

In one or more implementations, a generated user interface may include data on a metric for a first group of providers. The generated user interface may include a toggle. The toggle may allow the user to select a second group of providers. Values for the metric for the second group of providers may be auto-populated in the user interface. The values may be auto-populated in response to the selection of the second group of providers.

In one or more implementations, a generated user interface may include data on a first metric for one or more providers. The generated user interface may include a toggle. The toggle may allow the user to select a second metric. Values for the second metric for the one or more providers may be auto-populated in the user interface. The values may be auto-populated in response to the selection of the second metric.

In one or more implementations, the system may be configured to associate one or more activities with a case, a provider, and/or a group. The activities may be associated by a data analytics engine of the system.

In one or more implementations, the system may be configured to acquire location data. The location data may be acquired by a data acquisition engine of the system. The location data may be acquired from one or more location sensors. The location sensors may be part of one or more user devices of one or more caregivers.

In one or more implementations, the system may be configured to analyze acquired location data. The location data may be analyzed by an analytics engine of the system. The location data may be analyzed to identify one or more activities of one or more caregivers.

In one or more implementations, the system may be configured to acquire audiovisual data. The audiovisual data may be acquired by a data acquisition engine of the system. The audiovisual data may be acquired from one or more ambient sensors. The ambient sensors may be part of one or more user devices of one or more caregivers.

In one or more implementations, the system may be configured to analyze acquired audiovisual data. The audiovisual data may be analyzed by an analytics engine of the system. The audiovisual data may be analyzed to identify one or more activities of one or more caregivers.

In one or more implementations, the system may be configured to acquire textual data. The textual data may be acquired by a data acquisition engine of the system. The textual data may be acquired from one or more user interface devices. The user interface devices may be part of one or more user devices of one or more caregivers.

In one or more implementations, the system may be configured to analyze acquired textual data. The textual data may be analyzed by an analytics engine of the system. The textual data may be analyzed to identify one or more activities of one or more caregivers.

In one or more implementations, the system may be configured to acquire at least two of location data from a location sensor of a user device of a caregiver, audiovisual data from ambient sensors of the user device of the caregiver, and textual data from user interface devices of the user device of the caregiver. The location data, audiovisual data, and textual data may be acquired by a data analytics engine of the system.

In one or more implementations, the system may be configured to identify one or more activities of the caregiver using a combination of at least two of the acquired location data, audiovisual data, and textual data. The one or more activities may be identified by an analytics engine of the system.

One or more embodiments of the disclosure relate to a method. The method may comprise acquiring data on one or more activities of one or more caregivers in a healthcare organization. The method may also comprise generating comparison data on the caregivers in the healthcare organization. The method may moreover comprise generating one or more user interfaces. The one or more user interfaces may include data on activities of the provider. The one or more user interfaces may alternatively or additionally include comparison data comparing one or more metrics of multiple providers.

In one or more implementations, a generated user interface may include data on a metric for a first group of providers. The generated user interface may include a toggle. The toggle may allow the user to select a second group of providers. Values for the metric for the second group of providers may be auto-populated in the user interface. The values may be auto-populated in response to selection of the second group of providers.

In one or more implementations, a generated user interface may include data on a first metric for one or more providers. The generated user interface may include a toggle. The toggle may allow the user to select a second metric. Values for the second metric for the one or more providers may be auto-populated. Values may be auto-populated in the user interface in response to the selection of the second metric.

In one or more implementations, one or more activities may be associated with one or more of a case, a provider, and a group.

In one or more implementations, acquiring data on one or more activities of one or more caregivers in the healthcare organization may comprise acquiring location data from one or more location sensors. The location sensors may be part of one or more user devices of one or more caregivers. The acquired location data may be analyzed to identify one or more activities of one or more caregivers.

In one or more implementations, acquiring data on one or more activities of one or more caregivers in the healthcare organization may comprise acquiring audiovisual data. The audiovisual data may be acquired from one or more ambient sensors. The ambient sensors may be part of one or more user devices of one or more caregivers. The acquired audiovisual data may be analyzed to identify one or more activities of one or more caregivers.

In one or more implementations, acquiring data on one or more activities of one or more caregivers in the healthcare organization may comprise acquiring textual data. The textual data may be acquired from one or more user interface devices. The user interface devices may be part of one or more user devices of one or more caregivers. The acquired textual data may be analyzed to identify one or more activities of one or more caregivers.

In one or more implementations, acquiring data on one or more activities of one or more caregivers in the healthcare organization may comprise acquiring at least two of location data from a location sensor of a user device of a caregiver, audiovisual data from ambient sensors of the user device of the caregiver, and textual data from user interface devices of the user device of the caregiver. One or more activities of the caregiver may be identified using a combination of at least two of the acquired location data, audiovisual data, and textual data.

The foregoing is a summary of the disclosure and thus by necessity contains simplifications, generalizations and omissions of detail. Consequently, those skilled in the art will appreciate that the summary is illustrative only and is not intended to be in any way limiting.

Other aspects, inventive features, and advantages of the devices and/or processes described herein, as defined by the claims in related applications, will become apparent in the detailed description set forth herein and taken in conjunction with the accompanying drawings.

### BRIEF DESCRIPTION OF THE DRAWINGS

FIG. 1 is a simplified diagram of a healthcare information acquisition and presentation system, in accordance with at least some embodiments of the present disclosure.

FIG. 2A is an illustrative flowchart outlining steps of collecting, comparing, and presenting provider data using the system of FIG. 1, in accordance with at least some embodiments of the present disclosure.

FIG. 2B is an illustrative flowchart outlining steps of collecting and analyzing various data using the system of FIG. 1, in accordance with at least some embodiments of the present disclosure.

FIG. 3 is an illustrative user interface presenting provider data that may be acquired and analyzed via steps identified in FIG. 2, in accordance with at least some embodiments of the present disclosure.

FIG. 4 is an illustrative user interface presenting practice group data that may be acquired and analyzed via steps identified in FIG. 2, in accordance with at least some embodiments of the present disclosure.

FIG. 5 is an illustrative user interface in accordance with at least some embodiments of the present disclosure.

FIG. 6 is an illustrative user interface in accordance with at least some embodiments of the present disclosure.

FIG. 7 is an illustrative user interface in accordance with at least some embodiments of the present disclosure.

FIG. 8 is an illustrative user interface in accordance with at least some embodiments of the present disclosure.

FIG. 9 is an illustrative user interface in accordance with at least some embodiments of the present disclosure.

FIG. 10 is an illustrative user interface in accordance with at least some embodiments of the present disclosure.

FIG. 11 is an illustrative user interface in accordance with at least some embodiments of the present disclosure.

FIG. 12 is an illustrative user interface in accordance with at least some embodiments of the present disclosure.

FIG. 13 is an illustrative user interface in accordance with at least some embodiments of the present disclosure.

FIG. 14 is an illustrative user interface in accordance with at least some embodiments of the present disclosure.

### DETAILED DESCRIPTION

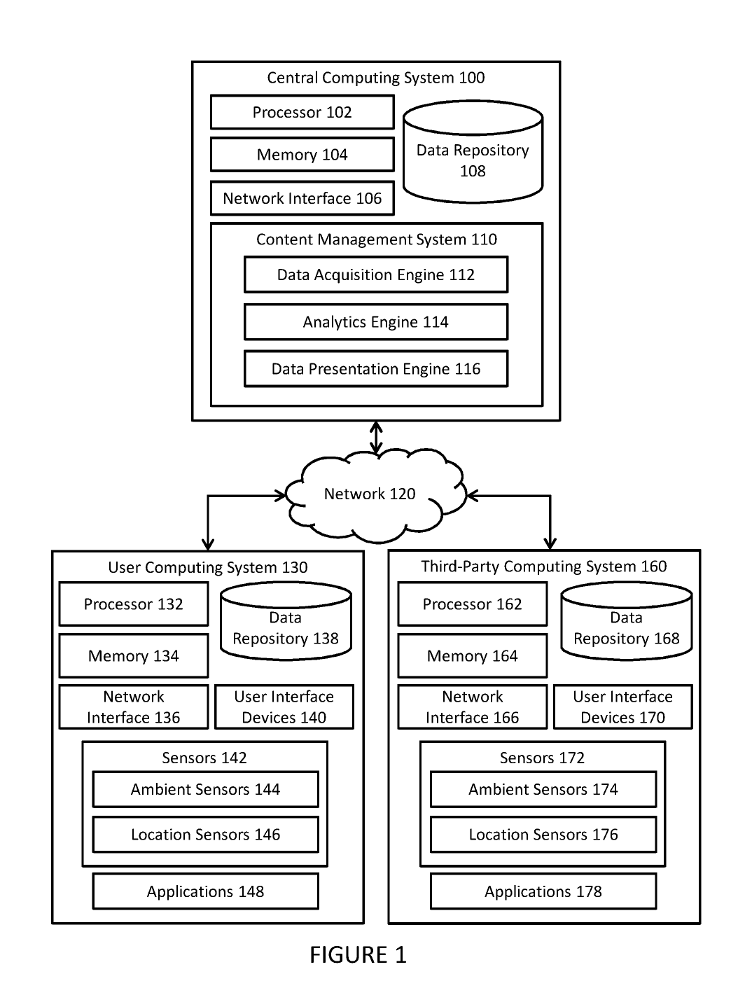

Referring to FIG. 1, an example system may include a central computing system 100 in communication, via a network 120, with one or more user computing systems 130 of one or more users, and with one or more third-party computing systems 160 of one or more third parties. Each computing system 100, 130, 160 may include, for example, one or more mobile computing devices (such as smartphones, tablets, laptops, etc.), non-mobile computing devices (such as desktop computers, workstations, servers, etc.), or a combination thereof. The mobile and non-mobile computing devices of each computing system 100, 130, 160 may be co-located or remote to each other. Computing systems 100, 130, 160 are communicably coupled to each other over the network 120, which may be any type of type of network. For example, the network may include wireless network interfaces (e.g., 802.11X, ZigBee, Bluetooth, Internet, etc.), wired network interfaces (e.g., Ethernet, USB, Thunderbolt, etc.), or any combination thereof to enable network connections between systems. The network 120 is structured to permit the exchange of data, values, instructions, messages, and the like between the computing systems 100, 130, 160 via such network connections.

The central computing system 100 includes processor 102, memory 104 with instructions executable by processor 102, network interface 106 for communicating with other computing devices and systems (e.g., via network 120), and a data repository 108 with one or more structured or unstructured databases capable of storing data related to healthcare providers. Similarly, the user computing system 130 (or each computing device therein) includes processor 132, memory 134 with instructions executable by processor 132, network interface 136 for communicating with other computing devices and systems (such as systems 100 and 160), and a data repository 138 with one or more structured or unstructured databases capable of storing data related to healthcare providers. And, the third-party computing system 160 (or each computing device therein) includes processor 162, memory 164 with instructions executable by processor 162, network interface 166 for communicating with other computing devices and systems, and a data repository 168 with one or more structured or unstructured databases capable of storing data related to healthcare providers.

The processor 102, 132, 162 may be implemented as a general-purpose processor, an application specific integrated circuit (ASIC), one or more field programmable gate arrays (FPGAs), a digital signal processor (DSP), a group of processing components, or other suitable electronic processing components structured to control the operation of the computing system 100, 130, 160, respectively. The memory 104, 134, 164 (e.g., RAM, ROM, NVRAM, Flash Memory, hard disk storage, etc.) may store data and/or computer code for facilitating at least some of the various processes described herein. In this regard, the memory 104, 134, 164 may store programming logic that, when executed by the processor 102, 132, 162, controls the operation of the computing system 100, 130, 160, respectively. The network interface 106, 136, 166 may be structured to allow the computing system 100, 130, 160, respectively, to communicate data to and from other devices either directly or indirectly. The computing systems 100 and 130 may be, for example, enterprise computing systems (with one or more computing devices) of an enterprise, and may receive and/or provide data stored in repository 108, 168 via a web browser (such as Google Chrome, Microsoft Edge, Internet Explorer, or other application for accessing information via the internet or other network) or other application (such as a native application for a mobile device that could be provided or authorized by the institution implementing system 100, 160) to facilitate communication with devices in systems 100, 130, 160.

The system 100 also includes content management system 110, which may be implemented using hardware, software (which may be stored on memory 104), or a combination thereof. The content management system 110 includes a data acquisition engine 112 configured to collect, retrieve, or otherwise acquire data on, for example, healthcare providers and their activities and organizations. An analytics engine 114 is configured to compare and analyze data that may have been acquired by the data acquisition engine 112. The data acquired via data acquisition engine 112 and/or obtained via analytics engine 114 may be stored in data repository 108. A data presentation engine 116 is configured to generate user interfaces and otherwise present data (e.g., via graphical user interfaces, verbal communication, tactile communication, etc.), and particularly data acquired via data acquisition engine 112 and/or obtained via analytics engine 114.

The user computing system 130, or one or more devices therein, may also include user interface devices 140, various sensors 142, such as ambient sensors 144 and location sensors 146, and applications 148. User interface devices 140 may include, for example, components that provide perceptible outputs (e.g., displays and light sources for visually-perceptible elements, a speaker for audible elements, and haptics for perceptible signaling via touch) and that allow the user to provide inputs (e.g., a touchscreen, stylus, force sensor for sensing pressure on a display screen, and biometric components such as a fingerprint reader, a heart monitor that detects cardiovascular signals, an iris scanner, and so forth). Ambient sensors 144 may include components that capture ambient sights and sounds (such as cameras and microphones) in the surroundings of the user computing system 130. One or more devices in user computing system 130 may include one or more location sensors 146 to enable the device to determine its location relative to, for example, other physical objects or relative to geographic locations. Example location sensors 146 include global positioning system (GPS) devices and other navigation and geolocation devices, digital compasses, gyroscopes and other orientation sensors, as well as proximity sensors or other sensors that allow the user device 130 to detect the presence and relative distance of nearby objects and devices. The user computing system 130 may additionally include applications 148, such as browsers and client applications provided or authorized by the entity implementing or administering the central computing system 100.

The third-party computing system 160, or one or more devices therein, may similarly also include user interface devices 170, various sensors 172, such as ambient sensors 174 and location sensors 176, and applications 178. Third-party devices may be mobile (e.g., smartphones, tablets, personal digital assistants) or relatively non-mobile (e.g., monitoring devices in sporting facilities, offices, and healthcare facilities like hospitals and clinics). Third-party computing systems 160 may include systems and devices associated with other healthcare providers and medical facilities, entities with patient records, e-mail servers, research portals, security systems, etc.

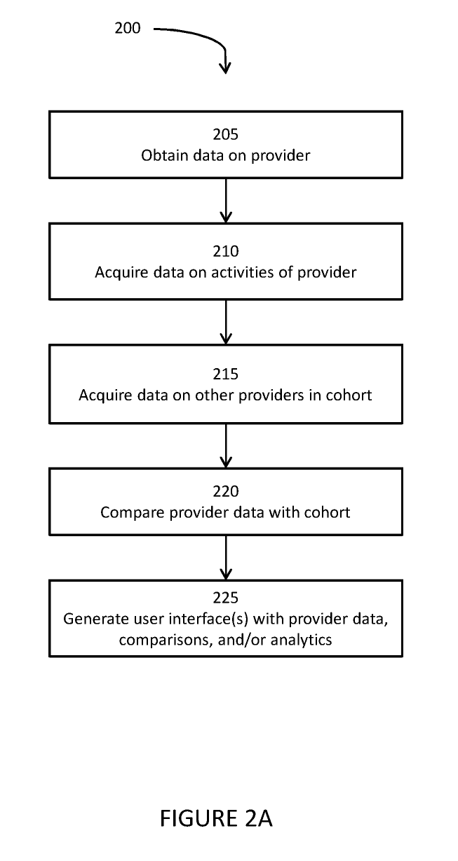

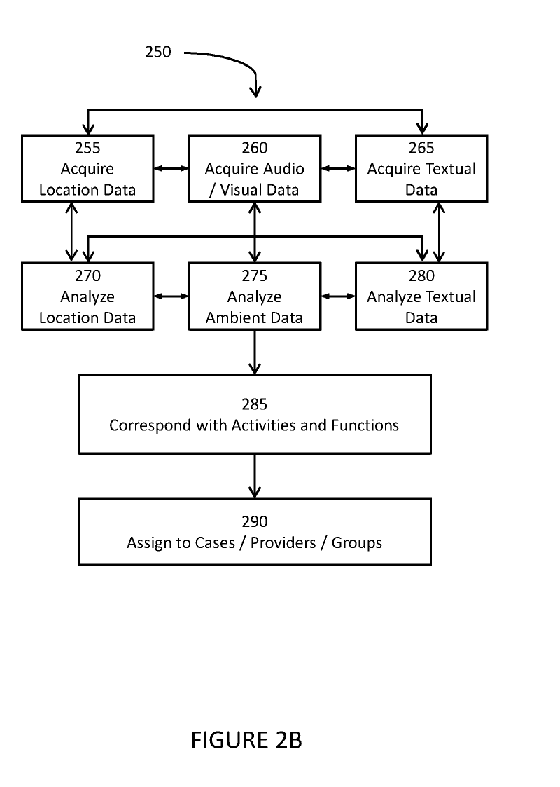

Referring to FIG. 2A, an example process 200 for collecting, comparing, and presenting data on healthcare providers and practice groups (e.g., clinics, hospitals, and other healthcare organizations) is presented. At 205, the central computing system 100 may obtain data on a provider, such as identifying information and information on the provider's roles, functions, etc. At 210, the system 100 may (via data acquisition engine 112) acquire data on the activities of a provider. This may be accomplished in one or more ways. For example, the system may log time spent by a provider drafting e-mails, composing text messages, speaking on the telephone (which may include use of video conferencing technology such as Skype), reviewing case files, reviewing literature (such as by accessing online journal articles and books), performing procedures, etc. In some implementations, this may be done automatically via one or more tracking applications running on one or more user devices of the provider. In other implementations, these data may be obtained from other sources, such as computing systems or applications that are separately maintained by the same entity or by other entities (e.g., using third-party computing system 160). As one example, data may be available via a service like SirenMD, and the central computing system 100 may interface or otherwise access data captured by or available to SirenMD. In yet other implementations, the data (such as time spent on certain activities) may be manually entered by the provider.

At 215, the central computing system 100 may acquire data on other providers in a cohort (such as providers in the same practice group at the same organization, providers in the same field but at multiple organizations and/or in the same or multiple geographical areas, etc.). This may be accomplished in any of the ways that data are captured for the provider at 210. For example, activities of other providers at the same or other organizations can be tracked via any of the devices and applications associated with any of the computing systems 100, 130, 160. In some implementations, relevant data may be retrieved from other databases (such as databases of governmental agencies or the databases of other healthcare organizations) if available.

At 220, the provider data is compared with the data on providers in the cohort via analytics engine 114. This may include calculating any statistics desired, such as mean, median, standard deviation, percentiles, etc. At 225, the system 100 may generate, via data presentation engine 116, one or more user interfaces with provider data and/or comparison data. Data may be presented visually, audibly, haptically, or any combination thereof.

Referring to FIG. 2B, an example process 250 for acquiring and analyzing data on the activities of healthcare providers and practice groups is presented. Process 255 may be performed, at least in part, via content management system 110 of central computing system 100. At 255, location data may be acquired using one or more location sensors 146, 176 of one or more devices in user computing system 130 and/or third-party computing system 160. At 260, audio or visual data may be acquired using ambient sensors 144, 174 of one or more devices in user computing system 130 and/or third-party computing system 160. Audio/visual data may include time spent in voice-only or video calls. At 265, textual data may be acquired using one or more user interface devices 140, 170 of one or more devices in user computing system 130 and/or third-party computing system 160.

At 270, location data may be analyzed to help identify where individuals have traveled and how long individuals have spent in each location. Location data may be analyzed to determine, for example, that a provider spent a certain amount of time in an examination room, in an office, in a clinic, in a moving car, in a locker room, in a patient's home, etc. At 275, ambient data may be analyzed to determine, for example, what topics, conditions, or patients are discussed, what may be occurring in a provider's surroundings, such as sports activity or treatment, who is present for a conversation or in a location via voice recognition, and what activities a provider is engaged in (such as examining a patient or administering a treatment). At 280, textual data may be analyzed to determine, for example, what topics, conditions, or patients are discussed and what is being annotated. The person or persons with which data is associated may be determined in part via biometric data, voice recognition of what is spoken by speakers in a conversation, facial recognition in video footage, etc.

At 285, the location, audiovisual data (images, video, sounds, etc.), and textual data may be determined to correspond with certain activities, functions, patients, treatments, etc. For example, a provider may be determined to be in the proximity of a certain patient, at a location corresponding with a schedule meeting, discussing a treatment option or research with a patient or other providers, taking notes related to a certain patient, condition, or treatment, and/or traveling (in a vehicle and/or on foot) to a location to meet a certain patient or for a certain function. A provider's location, surroundings, actions, conversations, communications, etc., may be analyzed to determine its relevance to particular cases, treatments, activities, etc. At 290, activities, and the time spent on each activity, may be assigned to particular cases, providers, groups, etc., based the analysis of the data and the activities and functions with which they correspond. Information acquired using multiple devices and sensors and analysis of data therefrom may be presented visually using graphical user interfaces or otherwise.

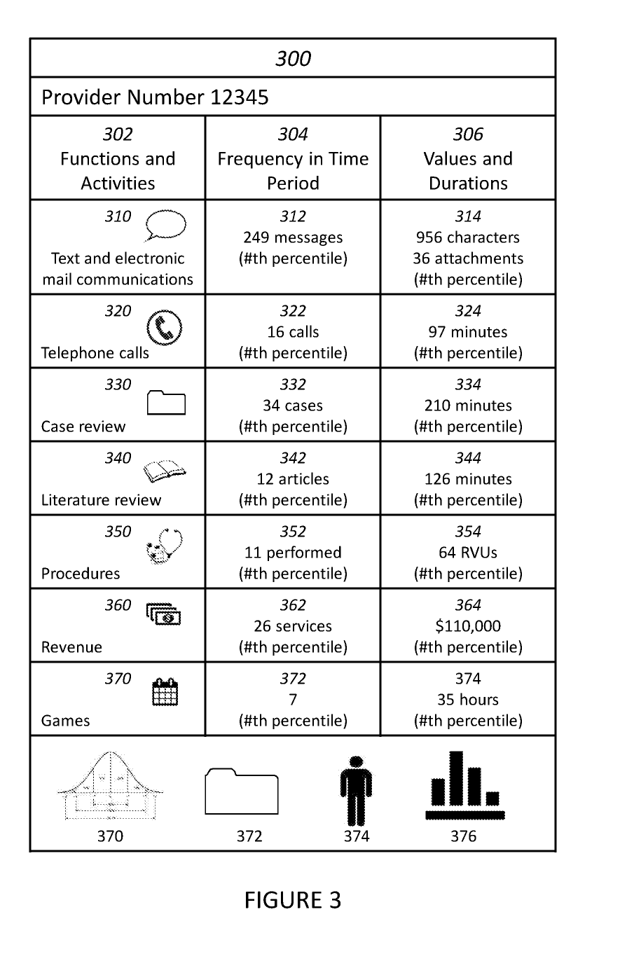

Referring to FIG. 3, an example user interface 300 (“dashboard” or “analytics dashboard”) is provided for a hypothetical healthcare provider (such as a physician, therapist, etc.) identified as “Provider Number 12345.” The interface 300 may be accessible to, for example, the provider or an organization with which the provider is affiliated (such as a practice group, clinic, or hospital). The interface 300 provides information on various functions and activities 302, and for each function/activity, provides a frequency of the function or activity occurring in a time period 304 (such as a day, week, month, quarter, or year). Also for each function and activity 302, the interface 300 provides values and durations 306.

The example interface 300 of FIG. 3 provides information on text and e-mail communications 310, and indicates a number of messages 312 (i.e., 249 messages), and total numbers of characters and/or attachments 314 (i.e., 956 characters and 36 attachments) for the messages 312. Similarly, interface 300 provides an entry for telephone calls 320, for which is provided the number of calls 322 (i.e., 16 calls), and the total number of minutes spent on those calls 322 (i.e., 97 minutes). Under case review 330, interface 300 provides information on the number of cases reviewed 332 (i.e., 34 cases), and the total time spent on the cases 334 (i.e., 210 minutes). Similarly, for literature review 340, interface 300 provides the number of articles reviewed 342 (i.e., 12 articles) and the total time spent on reviewing the articles 344 (i.e., 126 minutes).

Interface 300 also provides information on the procedures 350 (e.g., surgical interventions, treatments, etc.) indicating the number of procedures performed 352 (i.e., 11) and the RVUs 354 for the procedures performed (i.e., 64). Additionally, interface 300 provides the revenue 360 generated by the provider, indicating the number of services 362 performed (i.e., 26 services, of which the procedures 352 may be a part) and the revenue generated 364 (i.e., $110,000). Revenue data may be obtained, for example, from other systems (such as those of Epic), electronic medical records, etc.

Icon 370 (“Games”) refers to professional, collegiate, and amateur athletic sporting events. For example, the University of Miami Hurricanes football vs. the Duke University Blue Devils football would be one “Game.” Icon 372 (7) refers to the total number of Games during the period of time selected in 304 (Frequency in Time Period). Icon 374 (35 Hours) refers to the total number of hours accumulated by the caregiver at Games (370) during the selected period of time (304).

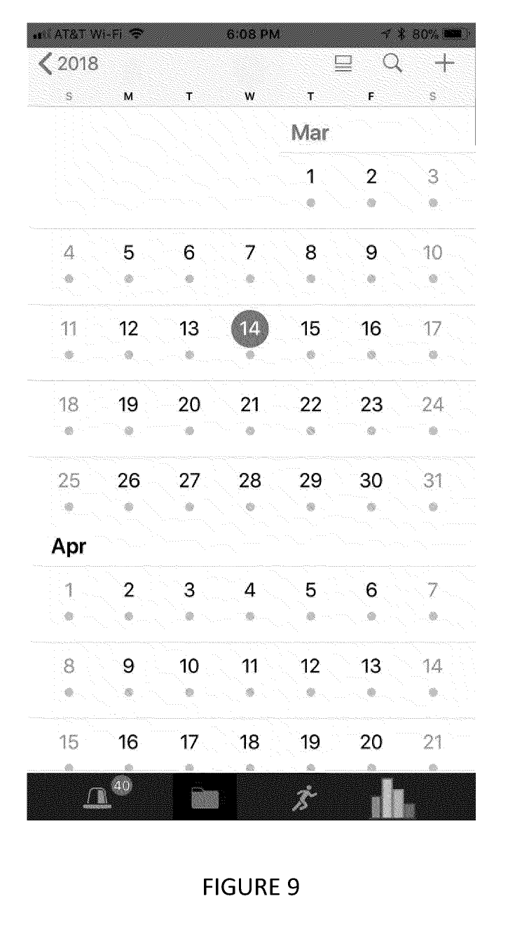

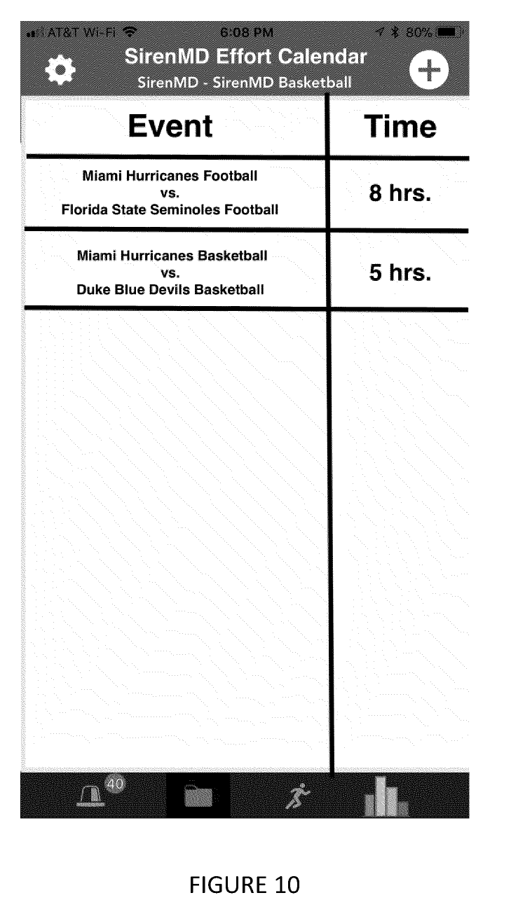

If a user selects icon 370 (Games), they may be brought to their “Calendar Page” (FIG. 9). If a user selects icon 372 or 374, they may be brought to their “SirenMD Effort Calendar” (FIG. 10).

Icon 376 (representing a generic graph) may be structured to allow users with access to a dashboard like the dashboard interface 300. Selecting icon 376 may provide a selection of available dashboards, such as a dashboard for the provider (such as Provider 12345), a dashboard for a cohort (like caregivers in the same specialty), a dashboard for providers in the organization, etc.

For certain metrics, information may be available on more than just the provider (i.e., on more than just Provider Number 12345). If data are available on other providers (e.g., other providers in the same specialty, practice, clinic, city, county, state, country, etc.) in a “cohort,” interface 300 can provide information that indicates how the provider compares with other providers in the cohort for various metrics. Interface 300 can thus provide, for example, statistics such as percentiles for each value under 304 (i.e., 312, 322, 332, 342, 352, 362, 372) and under 306 (i.e., 314, 324, 334, 344, 354, 364, 374). The statistics/comparison data can be anonymized except for authorized users, and the data may be withheld unless there is a certain minimum number (such as five or ten) of providers in a cohort if confidentiality is to be maintained.

In certain implementations, the information in interface 300 can be provided with more granularity. For example, text and e-mail communications 310 can be split up such that information on text messages is provided separately from (i.e., is not lumped in with) information on e-mail messages. For example, entry 312 can indicate the number of text messages and separately the number of e-mail messages. Similarly, entry 314 can provide the number of characters in text messages, the number and/or types of attachments (e.g., photos, videos, and audio files) in text messages, the number of characters in e-mail messages, and the number and/or types of attachments (e.g., media files and documents) in the e-mail messages. Further granularity can be achieved by, for example, indicating the numbers (e.g., phone numbers being called), addresses (e.g., e-mail addresses), individuals, case files, patients, articles and/or books, procedures and treatments, etc., that are being counted in interface 300.

Additional information/granularity can be provided in one interface or in multiple interfaces. For example, interface 300 could allow users to hover over, click on, touch, highlight, or otherwise select any element in interface 300 to access more detailed information on the element. In certain implementations, the additional information can be provided in the form of a pop-up screen that is visible so long as, for example, a cursor (e.g., a mouse cursor) or finger (e.g., placement of a finger on a touchscreen) is interacting with or in the vicinity of the element being detailed. In certain implementations, if the device being used includes a touchscreen, and the touchscreen has a pressure sensor (e.g., “3D Touch” on certain Apple devices), then pressing down on different elements to different degrees can provide different/additional information on certain topics. For example, touching an element at a low pressure level can be structured to, for example, redirect the user to another interface/page with additional details or provide a drop-down menu that allows the user to toggle between available views, whereas pressing down on an element at a relatively higher pressure level may provide more granular information, such as a list of the specific articles if “12 articles” is pressed or the specific procedures if “11 performed” is pressed, or quartiles if “#th percentile” is pressed for any of the elements with percentiles.

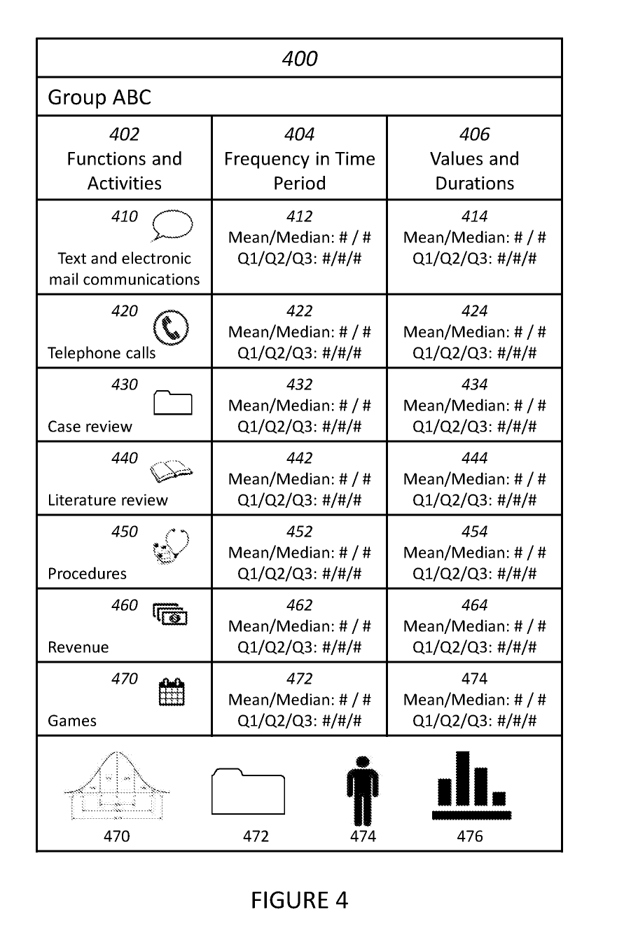

Referring to FIG. 4, an example user interface 400 is provided for a hypothetical healthcare organization (such as a practice group, clinic, hospital, etc.) identified as “Group ABC.” The interface 400 may be accessible to, for example, an administrator or other persons authorized by the healthcare organization, including providers at the organization if appropriate. As with interface 300, the interface 400 provides a column for information on various functions and activities 402, and for each function/activity, provides a column for information on frequency of the function or activity occurring in a time period 404 (such as a day, week, month, quarter, or year). Also for each function and activity 402, the interface 400 provides a column for values and durations 406.

The example interface 400 of FIG. 4 provides statistics such as mean, median, and the first, second, and third quartiles (Q1, Q2, and Q3, respectively) for multiple providers. Statistics are provided on text and e-mail communications 410 and total numbers of characters and/or attachments 414 for the messages 412. Similarly, interface 400 provides statistics for telephone calls 420 and the total number of minutes spent on those 422. Under case review 430, interface 400 provides statistics on the number of cases reviewed 432, and the total time spent on the cases 434. Similarly, for literature review 440, interface 400 provides statistics on the number of articles reviewed 442 and the total time spent on reviewing the articles 444. Interface 400 also provides statistics on procedures 450 (e.g., surgical interventions, treatments, etc.) and the RVUs 454 for the procedures performed. Additionally, interface 400 provides information on revenue 460 by providing statistics on number of services 462 performed and the revenue generated 464. Icons 470, 472, 474, and 476 perform the same functions as icons 370, 372, 374, and 376, respectively.

As with interface 300, additional information/granularity can be provided in one interface or in multiple interfaces. For example, interface 400 could allow users to hover over, click on, touch, highlight, or otherwise select any element in interface 400 to access more detailed information on the element. In certain implementations, the additional information can be provided in the form of a pop-up screen that is visible so long as, for example, a cursor (e.g., a mouse cursor) or finger (e.g., placement of a finger on a touchscreen) is interacting with or in the vicinity of the element being detailed. In certain implementations, if the device being used includes a touchscreen, and the touchscreen has a pressure sensor (e.g., “3D Touch” on certain Apple devices), then pressing down on different elements to different degrees can provide different/additional information on certain topics. For example, touching an element can redirect the user to another interface/page with additional details, whereas pressing down on an element may provide more granular information, such as the range of values or the specific values used to calculate a statistic.

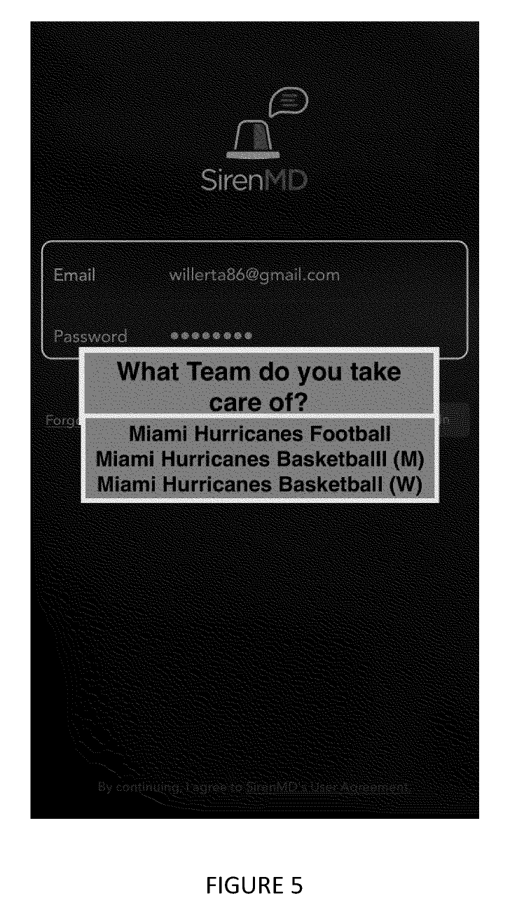

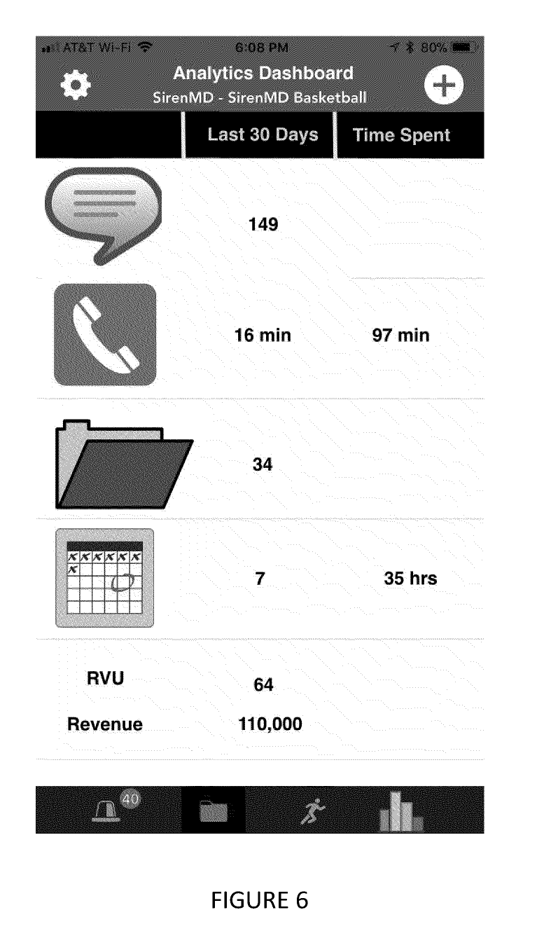

Referring to FIGS. 5-10, in certain implementations, the above functionalities may be incorporated into an existing system with information on providers, such as SirenMD, which is a technology platform for connecting trainers, physicians, and loved ones of athletes on a sports team. FIG. 5 shows an example user interface for logging into and entering the SirenMD platform for the first time. The user may be allowed to select which teams the providers of an organization serve. In various implementations, the selection will be confirmed (or pre-confirmed by an administrator) so that the provider may be granted privileges (and access to data) for the selected team. Referring to FIG. 6, an example user interface is provided, which may provide a link to an “analytics dashboard” (e.g., the graph icon at the bottom right of the user interface in FIGS. 3, 4, and 6-10) with more detailed information (such as the information in FIGS. 3 and 4).

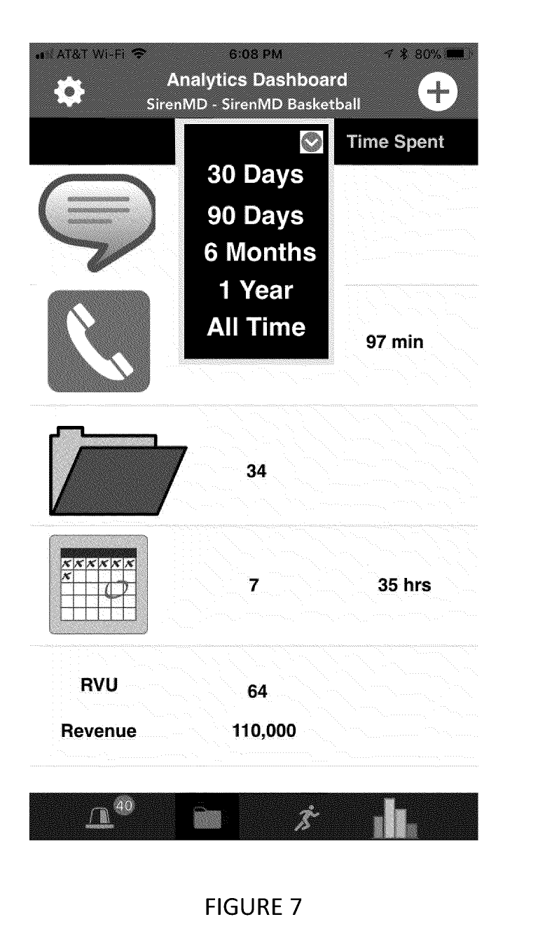

Referring to the example user interface in FIG. 7, a caregiver can view their analytics for a given time period using a toggle on the top tab. By touching or otherwise selecting the box with “Last 30 Days,” the user is able to choose other time periods for the same provider(s) for which data is provided in the interface. In this way, a user is able to toggle between metrics being displayed for the provider(s).

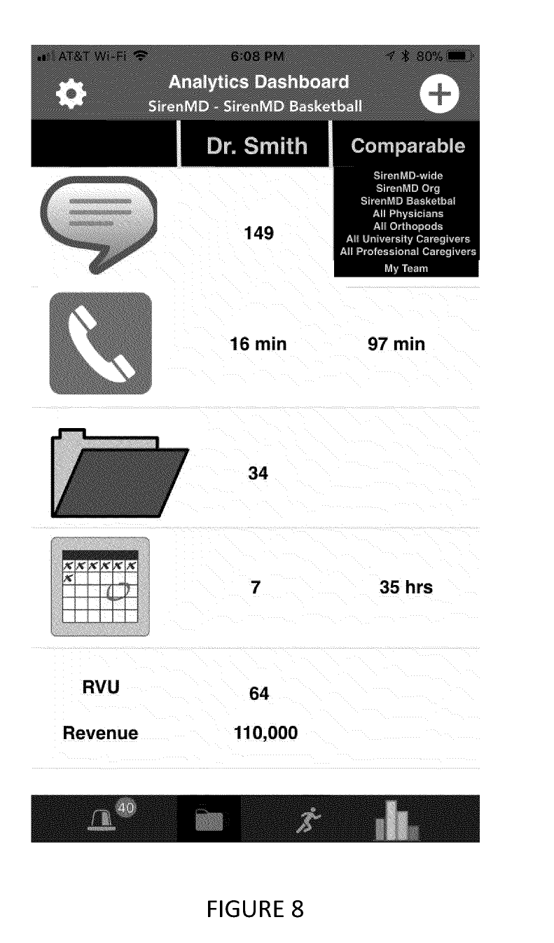

Referring to the example interface in FIG. 8, a caregiver can use an additional toggle to compare their performance to, for example: all registered caregivers (i.e., SirenMD-wide); all caregivers of a certain type in the caregiver's organization; all caregivers of a certain type on the caregiver's team; all caregivers in a specialty type; etc. When a user makes a selection to toggle between different available options in, the interface may automatically populate with data relevant to the selected item. Advantageously, in FIG. 8, the user selects “Time Spent,” and is provided with the option to access the same metric (i.e., “Time Spent”), but for other providers (or groups of providers). Once the other group of providers is selected, the values for the metric may be automatically updated without switching to another interface. This is distinguished from choosing to toggle between the metric that is displayed, but for the same provider (as is done in FIG. 7, with the user toggling between time period metrics without toggling provider group).

Referring to FIG. 9, when a caregiver taps on the calendar icon in FIG. 8, the caregiver may be directed to a page in which he or she can view a calendar of events. Based on the teams selected (and to which they are granted privileges), game schedule data may be incorporated in the calendar. Referring to FIG. 10, when a caregiver taps on the number of hours to the right of the calendar, he or she may be directed to a page that allows the caregiver to select the number of hours spent on a given event. Certain entries may be prepopulated with, for example, data acquired automatically using tracking and logging applications as discussed above.

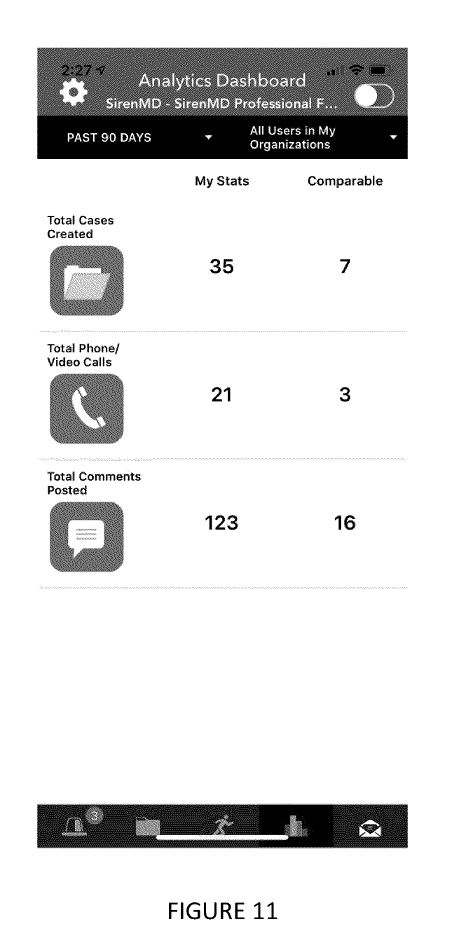

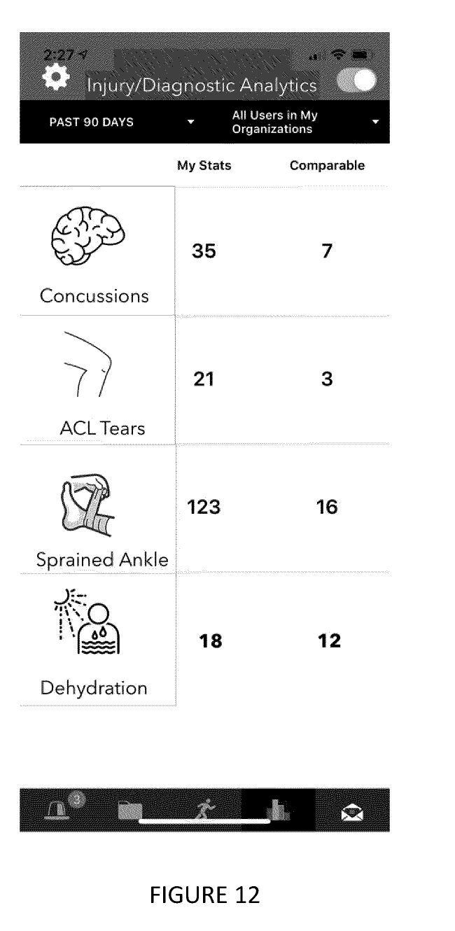

FIGS. 6 to 8 illustrate example productivity/activity analytics dashboards in accordance with potential embodiments. As can be seen in the upper-right corner of FIG. 11, a toggle switch (or other switch or selector) can be provided to allow a user to switch between a productivity/activity analytics dashboard and an injury/diagnostic analytics dashboard. In FIG. 11, the example toggle switch (which can be added to, e.g., the upper right corner or elsewhere in FIGS. 6 to 8) is switched to the left, and FIG. 11 is thus an example productivity/activity analytics dashboard. Various examples of potential injury/diagnostic analytics dashboards, with such a toggle switched to the right, are illustrated in FIGS. 12 to 14, in accordance with potential embodiments.

In injury/diagnostic analytics dashboards, a user may be presented with various injuries, conditions, diagnoses, etc., and the incidence of each over a given period of time. In FIG. 12, for example, the injuries listed are concussion, ACL tear, sprained ankle, and dehydration encountered over the “past 90 days.” For each injury, a user is provided with the number of times the corresponding injury was encountered in the selected time period under “my stats,” and a comparison number is provided, such as the average or median number of times the injury was encountered by other users in the selected cohort of “all users in my organizations” (e.g., all users in a team, a medical practice, or other organization). Alternatively or additionally, the user may be provided with other values, such as the range (highest and lowest numbers for each injury) for a selected time period and selected cohort. The injuries may be listed in any order desired, such as the most frequent injuries being listed at the top in decreasing order of frequency.

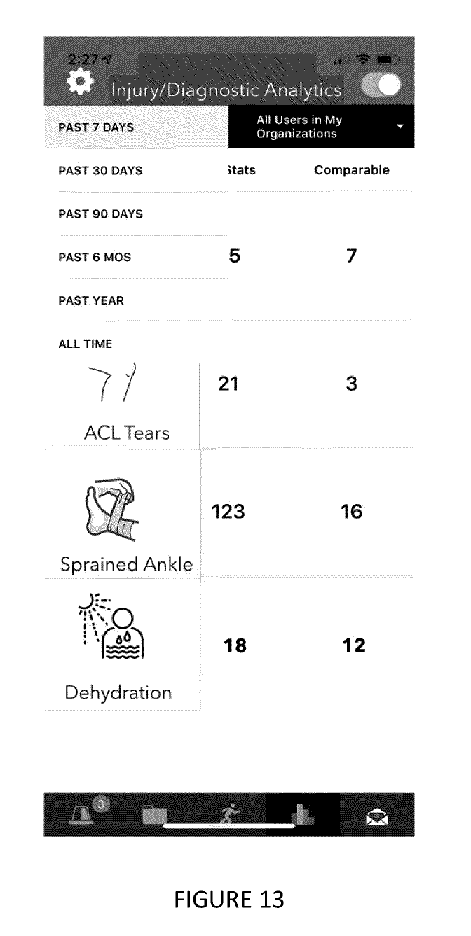

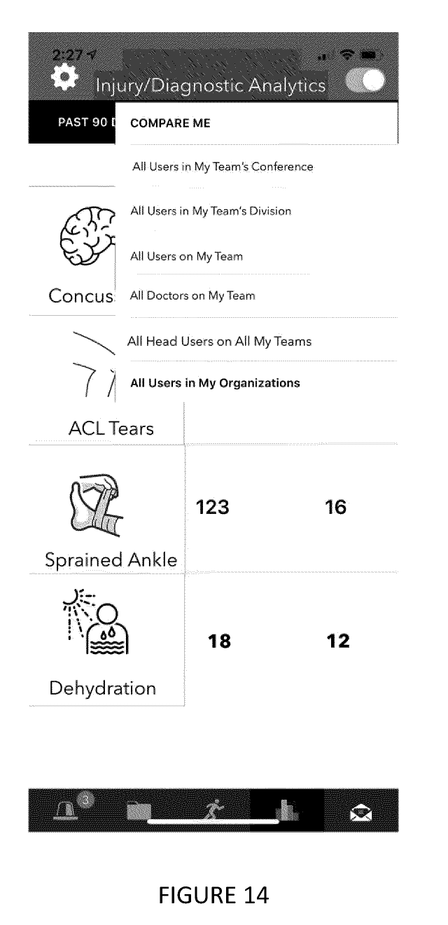

Referring to FIG. 13, the time period over which injuries are tallied can be switched to, for example, 7 days, 30 days, 90 days (selected in FIG. 12), 6 months, one year, or for all time (e.g., from the time the number of injuries began being tallied). Referring to FIG. 14, the cohort can be selected, such as “all users in my team's conference,” “all users in my team's division,” “all users on my team,” “all doctors on my team,” “all head users on all my teams,” and “all users in my organization.”

The disclosed approach enhances the ability to track, compare, and analyze activities, functions, and outcomes through capture and analysis of multiple types of data from multiple sensors and devices. The example user interfaces presented here provide an improved approach to summarizing and presenting information by better displaying a limited set of information to the user. Conventional interfaces require that a user makes a selection in a first (dedicated) interface, and view the items relevant to the selection in a second interface. By making selecting an item on the interface (such as “Time Spent”), the user is able to toggle between data without leaving the interface. The incorporation of game/event data in the SirenMD Calendar and SirenMD Effort Calendar allows each caregiver to accurately track his or her time spent at each event. This accuracy is important because, for example, while an athlete or spectator may need to allocate 4 hours for a football game, a team physician will log 8 hours. The breakdown of this 8 hours may be 2 hours before the game, 4 hours during the game, and 2 hours after the game. Recording this time accurately will not only provide motivation to caregivers (compare oneself to another group or individual), but may ultimately prove valuable for billing purposes.

Notwithstanding the embodiments described above in FIGS. 1-10, various modifications and inclusions to those embodiments are contemplated and considered within the scope of the present disclosure. Any of the operations described herein can be implemented as computer-readable instructions stored on a non-transitory computer-readable medium such as a computer memory.

It is also to be understood that the construction and arrangement of the elements of the systems and methods as shown in the representative embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed.

Accordingly, all such modifications are intended to be included within the scope of the present disclosure. Any means-plus-function clause is intended to cover the structures described herein as performing the recited function and not only structural equivalents but also equivalent structures. Other substitutions, modifications, changes, and omissions may be made in the design, operating conditions, and arrangement of the preferred and other illustrative embodiments without departing from scope of the present disclosure or from the scope of the appended claims.

Furthermore, functions and procedures described above may be performed by specialized equipment designed to perform the particular functions and procedures. The functions may also be performed by general-use equipment that executes commands related to the functions and procedures, or each function and procedure may be performed by a different piece of equipment with one piece of equipment serving as control or with a separate control device.

Moreover, although the figures show a specific order of method operations, the order of the operations may differ from what is depicted. Also, two or more operations may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection operations, processing operations, comparison operations, and decision operations.

What is claimed is:

1. A system, comprising:
a data acquisition engine configured to acquire data on one or more activities of one or more caregivers in a healthcare organization;
an analytics engine configured to generate comparison data on the caregivers in the healthcare organization; and
a data presentation engine configured to generate one or more user interfaces with data on activities of the provider and with comparison data comparing one or more metrics of multiple providers.

2. The system of claim 1, wherein a generated user interface includes data on a metric for a first group of providers, wherein the generated user interface includes a toggle to allow the user to select a second group of providers, wherein values for the metric for the second group of providers are auto-populated in the user interface in response to the selection of the second group of providers.

3. The system of claim 1, wherein a generated user interface includes data on a first metric for one or more providers, and wherein the generated user interface includes a toggle to allow the user to select a second metric, wherein values for the second metric for the one or more providers are auto-populated in the user interface in response to the selection of the second metric.

4. The system of claim 1, wherein the data analytics engine is further configured to associate the one or more activities to one or more of a case, a provider, and a group.

5. The system of claim 1, wherein the data acquisition engine is configured to acquire location data from one or more location sensors of one or more user devices of one or more caregivers.

6. The system of claim 4, wherein the analytics engine is further configured to analyze the acquired location data to identify one or more activities of one or more caregivers.

7. The system of claim 1, wherein the data acquisition engine is configured to acquire audiovisual data from one or more ambient sensors of one or more user devices of one or more caregivers.

8. The system of claim 7, wherein the analytics engine is further configured to analyze the acquired audiovisual data to identify one or more activities of one or more caregivers.

9. The system of claim 1, wherein the data acquisition engine is configured to acquire textual data from one or more user interface devices of one or more user devices of one or more caregivers.

10. The system of claim 9, wherein the analytics engine is further configured to analyze the acquired textual data to identify one or more activities of one or more caregivers.

11. The system of claim 1, wherein the data acquisition engine is configured to acquire at least two of:
location data from a location sensor of a user device of a caregiver;
audiovisual data from ambient sensors of the user device of the caregiver; and
textual data from user interface devices of the user device of the caregiver.

12. The system of claim 11, wherein the analytics engine is further configured to identify one or more activities of the caregiver using a combination of at least two of the acquired location data, audiovisual data, and textual data.

13. A method, comprising:
acquiring data on one or more activities of one or more caregivers in a healthcare organization;
generating comparison data on the caregivers in the healthcare organization; and
generating one or more user interfaces with data on activities of the provider and with comparison data comparing one or more metrics of multiple providers.

14. The method of claim 13, wherein a generated user interface includes data on a metric for a first group of providers, wherein the generated user interface includes a toggle to allow the user to select a second group of providers, wherein values for the metric for the second group of providers are auto-populated in the user interface in response to the selection of the second group of providers.

15. The method of claim 13, wherein a generated user interface includes data on a first metric for one or more providers, and wherein the generated user interface includes a toggle to allow the user to select a second metric, wherein values for the second metric for the one or more providers are auto-populated in the user interface in response to the selection of the second metric.

16. The method of claim 13, further comprising associating the one or more activities with one or more of a case, a provider, and a group.

17. The method of claim 13, wherein acquiring data on one or more activities of one or more caregivers in the healthcare organization comprises acquiring location data from one or more location sensors of one or more user devices of one or more caregivers, and wherein the method further comprises analyzing the acquired location data to identify one or more activities of one or more caregivers.

18. The method of claim 13, wherein acquiring data on one or more activities of one or more caregivers in the healthcare organization comprises acquiring audiovisual data from one or more ambient sensors of one or more user devices of one or more caregivers, and wherein the method further comprises analyzing the acquired audiovisual data to identify one or more activities of one or more caregivers.

19. The method of claim 13, wherein acquiring data on one or more activities of one or more caregivers in the healthcare organization comprises acquiring textual data from one or more user interface devices of one or more user devices of one or more caregivers, and wherein the method further comprises analyzing the acquired textual data to identify one or more activities of one or more caregivers.

20. The method of claim 13, wherein acquiring data on one or more activities of one or more caregivers in the healthcare organization comprises acquiring at least two of location data from a location sensor of a user device of a caregiver, audiovisual data from ambient sensors of the user device of the caregiver, and textual data from user interface devices of the user device of the caregiver, and wherein the method further comprises identifying one or more activities of the caregiver using a combination of at least two of the acquired location data, audiovisual data, and textual data.
